(19) United States
(12) Patent Application Publication
(10) Pub. No.: US 2019/0160103 A1
(43) Pub. Date: May 30, 2019

(54) PLATELET-RICH PLASMA COMPOSITION FOR TISSUE REPAIR AND REGENERATION

(71) Applicant: Stefania GARBIN, Lugano (CH)

(72) Inventor: Stefania GARBIN, Lugano (CH)

(21) Appl. No.: 16/069,637

(22) PCT Filed: Jul. 14, 2011
(86) PCT No.: PCT/EP2011/062045

§ 371 Date: Feb. 13, 2019

(51) Int. Cl. A61K 35/19; A61K 35/16; A61K 9/00; A61P 17/02; A61K 8/98; A61Q 19/08; A61Q 7/00

(52) U.S. Cl. CPC A61K 35/19; A61K 35/16; A61K 9/0014; A61M 1/3693; A61K 8/983; A61Q 19/08; A61Q 7/00; A61P 17/02

(57) ABSTRACT

The present invention relates to platelet-rich plasma (PRP) for epicutaneous use in a wide range of applications in the cosmetic and dermatological fields and to a functional bioactive composition suitable for topical use, which comprises said PRP and allows complete absorption into the skin, hair or scalp, and which has a regenerating action for relaxing expression wrinkles, a lifting effect for sustaining relaxed skin tissues, for scar reduction and wound healing. The present composition performs a regenerating and a repairing action on the skin cell layers, combining the benefits of increasing vitality and longevity of the skin, scalp and hair follicle, without the disadvantages of invasive techniques. The present composition is formulated to allow complete absorption of the PRP and guarantees the vital function of platelet growth factors while combining a gradual, more physiological releasing rate, and stimulating the recruiting of stem cells.

### FIELD OF THE INVENTION

The present invention relates to platelet-rich plasma (PRP) for epicutaneous use in a wide range of applications in the cosmetic and dermatological fields and to a composition suitable for topical use, which comprises said PRP which has a regenerating action for relaxing expression wrinkles, a lifting effect for sustaining relaxed skin tissues, for scar reduction and wound healing. The present composition performs a regenerating and a repairing action on the skin cell layers, increasing vitality and longevity of the skin, scalp and hair follicle.

### STATE OF THE ART

In the natural renewing cycle of human skin, cells are constantly being born, rising through the epidermal layers to the surface and are finally shed.

The cyclic renewal process of skin slows down as we age and other than the chronological turnover, our skin is subjected to stress due to photo-exposure, pollution, exposure to cigarette smoke and an unhealthy lifestyle. Ageing causes tissue repair and cell regeneration to slow down.

The skin aging process is marked by very distinct signs: the appearance of wrinkles, a variation in the skin pigmentation, loss of elasticity and compactness, and the relaxing of the tissues.

Skin aging indistinctly influences the three main skin structures: epidermis, dermal-epidermal junction and dermis. In particular, among the various factors which significantly participate in the skin aging process, is a reduction in the thickness of the dermis. The dermis is a connective tissue having a thickness of 3-4 mm, beneath the epidermis, consisting of a cell population dispersed in an abundant intercellular matrix. Typical dermal cells are fibroblasts, destined for the synthesis of collagen and elastic fibres. The former have the function of support and resistance, the elastic fibres ensure a correct elasticity of the skin whereas the intercellular matrix in which they are immersed has a strengthening function.

The three main skin structures influenced in skin ageing are those also involved in the process of wound healing.

This process is complex, involving several phases, such as the vascular reaction and blood clotting, inflammatory, proliferative and remodeling phases. These phases overlap and are partly mutually dependent, so that the sequence only approximately corresponds to the time course of wound healing.

Despite modern advances in wound closure techniques, devices and wound maintenance, there is still a critical need for new methods to enhance the healing process in order to achieve optimal outcomes. One of the promising yet clinically challenging areas of recent therapeutic development involves topical application of growth factors to enhance the normal healing process. Many of these new therapies have involved the provision of individual trophic factors with defined biological activities. They have a restricting growth factor release that is dependent on matrix loading, thus potentially limiting their overall ability to affect healing. Potentially more desirable for optimal wound healing would be therapies that augment the normal healing response by continuously supplying a mixture of factors mimicking the natural milieu.

Plasma is a very interesting “fluid tissue”. It is plenty of intriguing and fascinating molecules and cells that are responsible to regulate key process involved in tissue repair, including proliferation, chemotaxis, migration, cellular differentiation and extracellular matrix synthesis and remodeling.

Platelet-rich plasma (PRP) is blood plasma that has been enriched with platelets.

Blood is made of red blood cells, white blood cells, plasma, and platelets.

Platelets, initially known to be responsible for blood clotting, release a multitude of growth factors including Platelet-derived growth factor (PDGF), a potent chemotactic agent, and TGF beta, which stimulates the deposition of extracellular matrix. Both of these growth factors have been shown to play a significant role in the repair and regeneration of connective tissues.

Within PRP, the increased number of platelets delivers an increased number of growth factors to the topical area.

The seven principal known growth factors in PRP are: Platelet Delivered Growth Factors as (PDGFaa), (PDGFbb), (PDGFab,) Transforming Growth Factor beta-, (TGF-b), TGF-b2, Vascular Endothelial Growth Factor (VEGF), Epithelial Growth Factor (EGF).

As a concentrated source of platelets, PRP contains (and releases through degranulation) several different growth factors (cytokines) that stimulate tissue regeneration. PRP has received popular attention due to its use in treating sports injuries in professional athletes.

PRP therapy, involves centrifuging a person's blood until it contains a concentrated mix of plasma cells and growth factors and then injecting the resulting substance directly into the injured tissue.

The drawbacks of this therapy are many, starting from the pain of the injections to the need for medical intervention and the invasiveness of such a technique. There is a continuing need for the development of compositions which influence naturally occurring proteins of the human body such as growth factors, capable of stimulating cellular growth, proliferation and differentiation and therefore facilitate tissue repair and regeneration, and which are therefore useful in the processes of wound healing and to contrast skin ageing and relaxation.

The object of the present invention is therefore the development of improved compositions which are stable over time, easily prepared and which achieve the wound healing and tissue repair effects, which overcome the disadvantages related to invasive techniques such as fillers, injectables, and surgery which overcome the problems related to toxicity.

### SUMMARY OF THE INVENTION

The present invention concerns platelet-rich plasma (PRP) for the external topical treatment of wound healing and/or skin regeneration, wherein the platelet concentration is of at least 900,000 platelets/μl.

A further aspect of the present invention is a method for the preparation of the platelet-rich plasma according to the present invention, comprising the steps of:

a. collecting blood in a test tube;

b. separating the platelets by centrifugation;

c. mixing the platelets obtained in step b. with calcium chloride until the concentration of at least 900,000 platelets/μl is obtained.

A still further aspect of the present invention is a cosmetic composition comprising an effective amount of the platelet-rich plasma according to the present invention and cosmetically acceptable excipients.

In a further aspect the invention provides the use of the cosmetic composition, as an adjuvant in skin repair and regeneration and/or skin protection and/or preventing hair loss and/or for promoting the physiological growth of hair.

In a further embodiment, the invention provides a pharmaceutical composition, comprising an effective amount of the platelet-rich plasma according to the present invention, and pharmaceutically acceptable excipients.

In a still further embodiment, the invention provides a pharmaceutical composition for use in the treatment of wound healing and/or traumatic injuries, comprising an effective amount of the platelet-rich plasma according to the present invention, and pharmaceutically acceptable excipients.

A further aspect of the present invention relates to a sterile container, suitable for receiving the pharmaceutical composition.

A still further aspect of the present invention is a sterile container, suitable for receiving the cosmetic composition.

As will be further described in the detailed description of the invention, the PRP of the present invention have the advantage of being useful in a wide range of applications in the cosmetic and dermatological fields.

### BRIEF DESCRIPTION OF THE DRAWINGS

The characteristics and advantages of the present invention will be apparent from the detailed description reported below, from the Examples given for illustrative and non-limiting purposes, and from the annexed FIGS. 1-2, wherein:

FIG. 1: shows a schematic representation of three containers for the compositions according to Example 3, a face cream container, a body cream container and a hair gel container. The containers may be conveniently made of a biomaterial or for example of a porous polyolefin polymer material, to allow oxygen permeability in order to guarantee the PRP stability and vitality. The containers are sealed, maintain product sterility and impede direct access to the composition.

FIG. 2: shows a black and white photocopy of the color photograph of the container of FIG. 1, during PRP transfer.

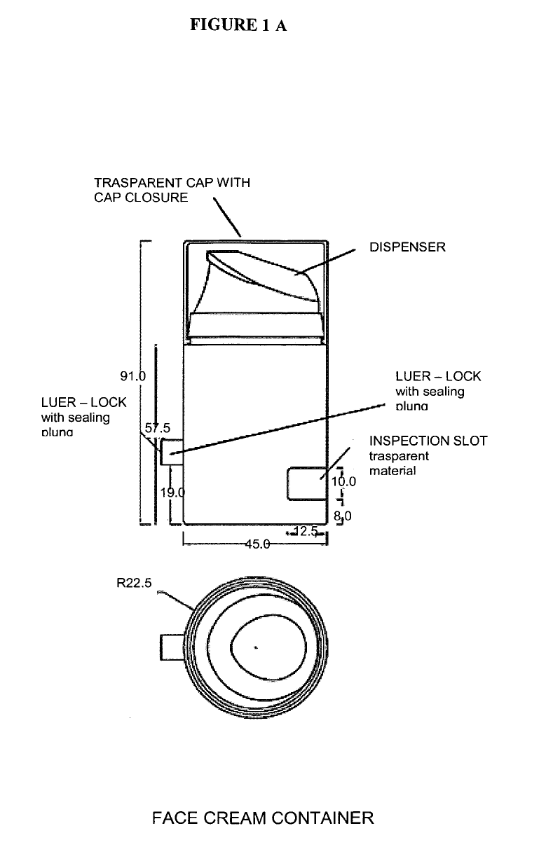

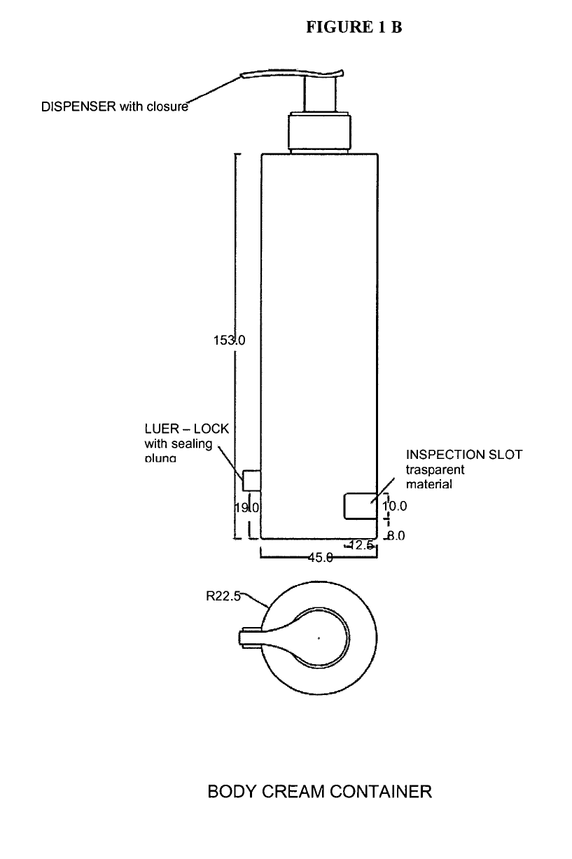

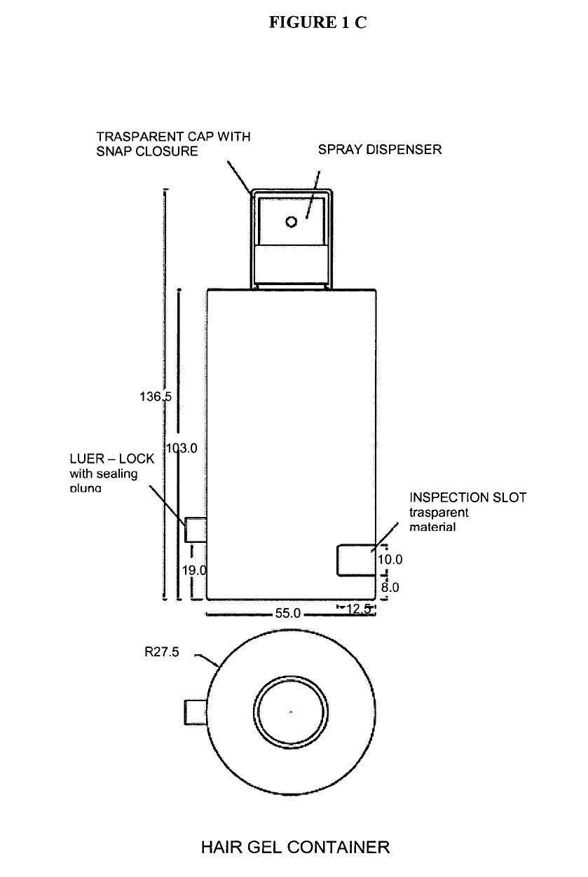

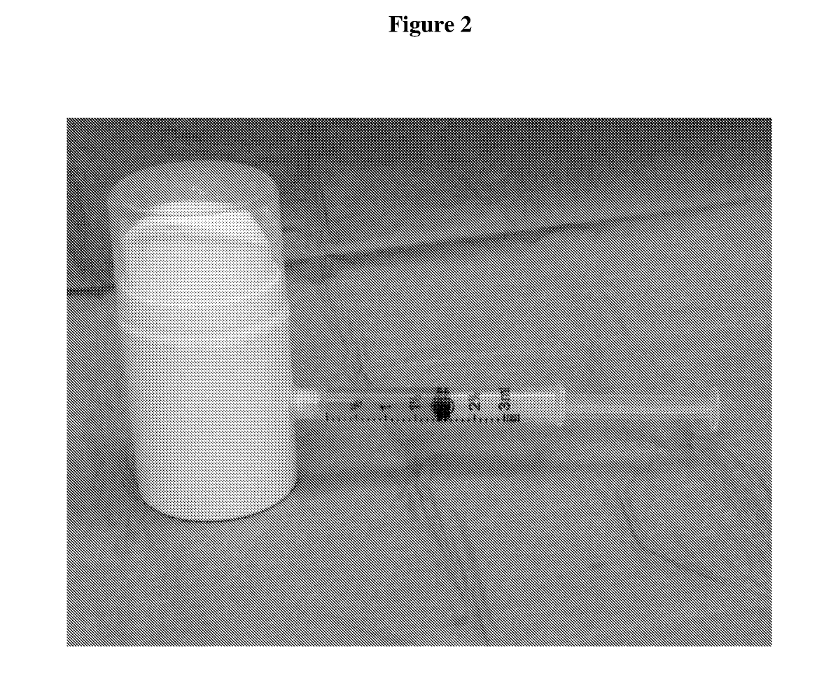

### DETAILED DESCRIPTION OF THE INVENTION

The present invention concerns platelet-rich plasma (PRP) for the external topical treatment of wound healing and/or skin regeneration, wherein the platelet concentration is of at least 900,000 platelets/μl.

For the purposes of the present invention by external topical administration an epicutaneous or an application onto the skin, hair, mucous membranes or scalp is intended.

Platelets are non-nuclear cellular fragments derived from megakaryocytes in the bone marrow through controlled cellular fragmentation.

They are specialized secretory cells that release the contents of their intracellular granules in response to activation. Platelets contain a complete array of pre-synthesized protein molecules, among which the high presence of cytoskeletal proteins, signalling proteins, membrane proteins, protein-processing proteins, and cytoskeleton regulatory proteins is noted. When platelets are activated, they exocytose the granules.

Platelet secretory granules contain growth factors (GFs), coagulation proteins, cell-activating molecules, cytokines, integrins, which are synthesized in megakaryocytes and packaged into the granules though vesicle trafficking processes. Three major storage compartments in platelets are alpha granules, dense granules, and lysosomes. The majority of the substance are contained in alpha granules.

Due to the large stores of cytokines and growth factors that are normally released during clot formation at wound sites, platelets, and consequently PRP (a concentrate of platelets from plasma), release cytokines and growth factors in the neighboring milieu.

The PRP according to the present invention thus has the advantage of being a concentrate of platelet growth factors, and the convenience of a topical application which is a non invasive, fast, and simple procedure. Invasive techniques, such as painful injections of PRP, on the contrary, need medical assistance.

Further, topical use of platelet-enriched preparations advantageously stimulate the natural healing cascade and tissue regeneration by a supra-physiological release of platelet derived factors directly in the site of treatment.

In a preferred aspect, the PRP according to the present invention is a PRP wherein the platelets are autologous and therefore the donor and the recipient are the same person. The autologous PRP has the advantages of deriving from the exact biological background, and therefore the PRP derived growth factors are in the most favorable situation for inducing skin, scalp and hair regeneration.

Furthermore, being the PRP an organic fluid belonging to the donor, a totally safe use is also guaranteed.

The PRP according to the present invention can be also a PRP wherein the platelets are allogenic, allogenic single donor platelets units from the blood bank that were ABO and RhD matched, virus checked, leukocyte depleted, irradiated and activated by human thrombin.

The number of platelets in the blood is referred to as the platelet count and is normally between 150,000 to 450,000 per microliter of blood.

The platelet growth factors content is quite variable among individuals and it is in not necessarily proportional to the platelet count.

A further aspect of the present invention is a method for the preparation of the platelet-rich plasma according to the present invention, comprising the steps of:

a. collecting blood in a test tube;

b. separating the platelets by centrifugation;

c. mixing the platelets obtained in step b. with calcium chloride until the concentration of at least 900,000 platelets/μl is obtained.

Advantageously the method according to the present invention can be performed with further steps.

In a preferred embodiment the method according to the present invention comprises a step d. of adding the mixture obtained in step c. to a cosmetically acceptable carrier or to a pharmaceutically acceptable carrier.

Platelets are extremely sensitive to any kind of process induced stress, from blood extraction to PRP composition production. Thus the amount of platelet-derived factors available at the end of the manipulation process depends on cumulative effects over platelets, starting from phlebotomy and ending with the formation of a PRP composition.

The platelet concentration of at least 900,000 platelets/μl, which corresponds totwoto six folds platelet concentration in whole blood, preferably 1,000,000 platelets/μl, more preferably 1,500,000 platelets/μl has been proven as most effective.

A still further aspect of the present invention is a cosmetic composition comprising an effective amount of the PRP according to the present invention, and cosmetically acceptable excipients.

In a further embodiment the PRP concentration of the cosmetic composition according to the present invention, is from 0.1 to 5.0% (w/w) of the total weight of the composition.

In a still further embodiment the cosmetic composition according to the present invention, is in a form selected from the group consisting of a solution, suspension, emulsion, ointment, foam, paste, gel, cream, lotion, powder, soap, surfactant-containing cleansing, oil, and spray, advantageously the cosmetic composition can be impregnated or made part of a bandage. The bandage can be a surgical dressing, a plaster bandage, an adhesive bandage or a gauze.

In a further aspect the invention provides the use of the cosmetic composition, as an adjuvant in skin repair and regeneration and/or skin protection and/or preventing hair loss and/or for promoting the physiological growth of hair.

Advantageously, the cosmetic composition may be used as an adjuvant for preventing or reducing photoaging and/or skin oxidative stress and/or regenerating the hair bulb cells and scalp.

This cosmetic composition according to the present invention combines the benefits of an enhanced wound healing and regeneration, without the disadvantages of invasive techniques.

The cosmetic composition according to the present invention comprises a functional bioactive emulsion, a vehicle exclusively created to grant a perfect mixture with platelets-derived growth factors, to allow their complete absorption into the skin or scalp.

The cosmetic composition according to the present invention has been advantageously seen useful as an adjuvant in body shape modeling, and in the treatment of cellulite as well as for reducing adipose accumulation.

The particular cosmetic composition is formulated to allow complete absorption of the PRP by guaranteeing, at the same time, the longevity, maintenance and function of platelet growth factors mixed in and released, stimulating the recruiting of stem cells.

The composition does not only allow treatment of ulcerated, burned or wounded tissues but also normal uninjured skin or scalp. The cosmetic composition is an adjuvant in ageing skin, spots, wrinkles, acne scars, post surgical scars, stretch marks, hair loss, as well as other aesthetic defects, yielding all the benefits without patient discomfort.

The cosmetic composition according to the present invention can advantageously be enriched with one or more of: monocytes, stem cells, genetherapy products, vitamins, retinol palmitate, tocoferil acetate, sodium ascorbil phosphate, D-panthenol, peptides, recombinant growth factors, micronized human-identical hormones, aminoacids, phyto-extracts, anti-oxidants, lipoic acid, DMAE, collagen, GAG, hyaluronic acid, proteoglycans, adenine, guanine, cytosine, thimine, trace elements, minerals, proteases, ceramides, polisaccarides, algae and marine extracts.

The cosmetic composition according to the present invention comprises the following formulation:

| | | |
|---|---|---|
| Platelet-rich plasma | Regenerating, healing | * |
| Caprilyc Capric | Fluid lipid | 10% |
| Tryglyceride | | |
| Glyceryl Stearate, | Lipo Amminoacidic emulsifier | 6% |
| Stearic acid, Cetearyl | | |
| Alcohol, Sodium | | |
| Lauroyl Glutamate | | |
| Cetyl Palmitate | Solid lipid, consistency factor | 5% |
| Cetearyl Alcohol | Solid lipid, consistency factor | 3% |
| Glycerin, Sodium | Ecocertified preserver | 3% |
| Anisate, Sodium | | |
| Levulinate | | |
| Sorbitol | Humectant | 5% |
| Glyceril Caprilate | Ecocertified preserver | 3% |
| Demineralized, sterile | Diluent | as required |
| water | | up to 100% |

* The Platelet-rich plasma according to the invention has the following concentrations in the composition for cosmetic use: the platelet-rich plasma concentration is from 0.1 to 5.0% (w/w) of the total weight of the composition;

The cosmetic composition according to the present invention formulated as a gel comprises the following formulation:

| | | |
|---|---|---|
| Platelet-rich plasma | Regenerating, healing | * |
| CarbopolETD20 | Synthetic polymer, jellifying | 1% |
| AminoMethyl | Neutralizer, for carbopol | 1% |
| propandiol | salification | |
| Alga Laminaria | Lipolitic | 1% |
| extract | | |
| Ivy extract | Fleboactive, Capillary protector | 1% |
| Caffein | Lipolitic | 1% |
| Sorbitol | Humectant | 5% |
| Citrus Amara Extract, | Modeling active principle | 3% |
| Lecithin | | |
| Glycerin, Sodium | Ecocertified preserver | 3% |
| Anisate, Sodium | | |
| Levulinate | | |
| Glyceril Caprilate, | Ecocertified preserver | 3% |
| Disodium EDTA | Conjugator | 0.1 |
| Pentylene Glycol | Ecocertified preserver | 0.50% |
| Demineralized, sterile | Diluent | as required |
| water | | up to 100% |

* platelet-rich plasma concentration is from 0.1 to 5.0% (w/w) of the total weight of the composition.

In a further embodiment, the invention provides a pharmaceutical composition, comprising an effective amount of the platelet-rich plasma according to the present invention, and pharmaceutically acceptable excipients.

In a further embodiment the PRP concentration of the pharmaceutical composition according to the present invention, is from 5.1 to 50.0% (w/w) of the total weight of the composition.

In a still further embodiment the pharmaceutical composition according to the present invention, is in a form selected from the group consisting of a solution, suspension, emulsion, ointment, foam, paste, gel, cream, lotion, powder, soap, surfactant-containing cleansing, oil, serum, drops, liposomes, nanoparticles, nanoboots and spray; advantageously the pharmaceutical composition can be impregnated or made part of a bandage. The bandage can be a surgical dressing, a plaster bandage, an adhesive bandage or a gauze.

A further aspect or the present invention regards a pharmaceutical composition for use in the treatment of wound healing and/or traumatic injuries, comprising an effective amount of the platelet-rich plasma according to the present invention, and pharmaceutically acceptable excipients.

In a preferred embodiment, the invention provides a pharmaceutical composition according to the present invention, for use in the treatment of a wound selected from the group consisting of a burn, a cut, an incision, and abrasion, a laceration or a contusion.

The value of PRP application on burns stimulates angiogenesis, promoting vascular in-growth and fibroblast proliferation. In addition, PRP functions as haemostatic by forming a fibrin clot.

PRP has advantage of enhancing wound-healing in both soft and hard tissue and in the treatment of acne scars.

In a more preferred embodiment, the invention provides a pharmaceutical composition for use in the treatment of traumatic injuries selected from the group consisting of chronic tendinopathies, acute ligamentous injuries, muscle injuries, bone injuries, rheumatic osteoarthritis, rheumatoid disease, autoimmune diseases, articular cartilage lesions, joint degenerations, maxillofacial injuries and osteoporosis.

A further aspect of the present invention relates to a sterile container, suitable for receiving the pharmaceutical composition.

The pharmaceutical composition according to the present invention can advantageously be enriched with one or more of: monocytes, stem cells, gene therapy products, vitamins, palmitate retinol, tocoferil acetate, sodium ascorbil phosphate, D-panthenol, peptides, recombinant growth factors, micronized human-identical hormones, aminoacids, phyto-extracts, anti-oxidants, lipoic acid, DMAE, collagen, GAG, hyaluronic acid, proteoglycans, adenine, guanine, cytosine, thimine, trace elements, minerals, proteases, ceramides, polisaccarides, algae and marine extracts.

The pharmaceutical composition according to the present invention formulated as a cream comprises the following formulation:

| | | |
|---|---|---|
| Platelet-rich plasma | Regenerating, healing | * |
| Caprilyc Capric | Fluid lipid | 10% |
| Tryglyceride | | |
| Glyceryl | Lipo Amminoacidic emulsifier | 6% |
| Stearate, Stearic acid, | | |
| Cetearyl Alcohol, | | |
| Sodium Lauroyl | | |
| Glutamate | | |
| Cetyl Palmitate | Solid lipid, consistency factor | 5% |
| Cetearyl Alcohol | Solid lipid, consistency factor | 3% |
| Glycerin, Sodium | Ecocertified preserver | 3% |
| Anisate, Sodium | | |
| Levulinate | | |
| Sorbitol | Humectant | 5% |
| Glyceril Caprilate | Ecocertified preserver | 3% |
| Demineralized, sterile | Diluent | as required |
| water | | up to 100% |

* The Platelet-rich plasma according to the invention has the following concentrations in the composition pharmaceutical use: the platelet-rich plasma concentration is from 5.1 to 50.0% (w/w) of the total weight of the composition.

The pharmaceutical composition according to the present invention formulated as a gel comprises the following formulation:

| | | |
|---|---|---|
| Platelet-rich plasma | Regenerating, healing | * |
| CarbopolETD20 | Synthetic polymer, jellifying | 1% |
| AminoMethyl | Neutralizer, for carbopol | 1% |
| propandiol | salification | |
| Alga Laminaria | Lipolitic | 1% |
| extract | | |
| Ivy extract | Fleboactive, Capillary protector | 1% |
| Caffein | Lipolitic | 1% |
| Sorbitol | Humectant | 5% |
| Citrus Amara Extract, | Modeling active principle | 3% |
| Lecithin | | |
| Glycerin, Sodium | Ecocertified preserver | 3% |
| Anisate, Sodium | | |
| Levulinate | | |
| Glyceril Caprilate | Ecocertified preserver | 3% |
| Disodium EDTA | Conjugator | 0.1 |
| Pentylene Glycol | Ecocertified preserver | 0.50% |
| Demineralized, sterile | Diluent | as required |
| water | | up to 100% |

* platelet-rich plasma concentration is from 5.1 to 50.0% (w/w) of the total weight of the composition.

A still further aspect of the present invention is a sterile container, suitable for receiving the cosmetic composition

The containers may be conveniently made of a biomaterial or for example of a porous polyolefin polymer material, to allow oxygen permeability in order to guarantee the PRP stability and vitality. The container is sealed, maintains product sterility and impedes direct access to the composition.

### EXAMPLES

### Example 1

Platelet Separation from Whole Blood

Before collecting blood, every patient made a complete blood examination including viral Hepatitis, HIV, Creutzfeldt-Jakob disease (CJD) and was analysed from a clinical point of view.

Exclusion criteria were: history of cancers, pregnancy, anemia, preexisting coagulation defects including trombocitopenia, hypofibrinoginemia, anticoagulation medications, ipersensitivity to bovine product which may be used for platelets activation.

After obtaining informed consent from the patients, an 8 ml blood sample was aspirated and collected in one 8 ml test tube. This tube was equipped with a separator, which centrifugally separates red and white platelet-rich plasma.

Each test tube was centrifuged at 3,000 rpm (1500 g) for seven minutes.

After centrifuging, the different cell components of the blood will be seen clearly separated in the test tube.

The platelet concentrate settles on the surface. Approximately 4 ml of concentrated PRP was obtained in the test tube.

With the 2 ml syringe and the transfer device, 0.9 ml of PRP was taken from the test tube and mixed with 0.1 ml CaCl₂.

The PRP was homogenized by gently turning the test tube upside down a few times. The resulting product is the PRP cell concentrate.

This procedure was repeated as often as necessary.

### Example 2

Mixture of the PRP with a Bio-Active Cream or Gel Carrier

Under a laminar flow hood, at least 2 ml of the PRP obtained according to Example 1 was added to a container enclosing 50 ml of the bio-active cream or gel carrier. In particular such a container may be structured in such a way that the PRP is conveyed through a practical and simple Luer-lock closure, in order to grant to the maximum extent of sterility of the final product. The complete procedure, from the blood collection to the preparation of the composition, is a sterile “closed system”.

The complete final product was gently shaken. The container conveniently has an inspection slot in order to be able to view the product.

The composition was seen to be stable at room temperature a temperature which mimics the physiological body temperature and prolongs the platelets growth factor release.

The composition was stable and functional for a period of at least three months.

### Example 3

Formulation of a Composition Comprising PRP According to the Invention

Cream

| Ingredient | Function | Percentage |
|---|---|---|
| Platelet-rich plasma | Regenerating, healing | * |
| Caprilyc Capric | Fluid lipid | 10% |
| Tryglyceride | | |
| Glyceryl | Lipo Amminoacidic emulsifier | 6% |
| Stearate, Stearic acid, | | |
| Cetearyl Alcohol, | | |
| Sodium Lauroyl | | |
| Glutamate | | |
| Cetyl Palmitate | Solid lipid, consistency factor | 5% |
| Cetearyl Alcohol | Solid lipid, consistency factor | 3% |
| Glycerin, Sodium | Ecocertified preserver | 3% |
| Anisate, Sodium | | |
| Levulinate | | |
| Sorbitol | Humectant | 5% |
| Glyceril Caprilate, | Ecocertified preserver | 3% |
| Demineralized, sterile | Diluent | as required |
| water | | up to 100% |

* The Platelet-rich plasma according to the invention has the following concentrations in the compositions: When the composition is for cosmetic use: the platelet-rich plasma concentration is from 0.1 to 5.0% (w/w) of the total weight of the composition; When the composition is for pharmaceutical use: the platelet-rich plasma concentration is from 5.1 to 50.0% (w/w)of the total weight of the composition.

The emulsion structure was studied and optimized to obtain a functional cream, which is not too oily and permits the maximum vitality to the platelets.

It has characteristics of perfect skin compatibility and skin similarity with structures available in the skin tissue, as well as in the body.

This cream also moisturizes, replenishes and stabilizes the lipid content.

This was achieved by adding natural emolients, emulsifiers, surfactants and humectant sorbitol.

Gel

| Ingredient | Function | Percentage |
|---|---|---|
| Platelet-rich plasma | Regenerating, healing | * |
| CarbopolETD20 | Synthetic polymer, jellifying | 1% |
| AminoMethyl | Neutralizer, for carbopol | 1% |
| propandiol | salification | |
| Alga Laminaria | Lipolitic | 1% |
| extract | | |
| Ivy extract | Fleboactive, Capillary protector | 1% |
| Caffein | Lipolitic | 1% |
| Sorbitol | Humectant | 5% |
| Citrus Amara Extract, | Modeling active principle | 3% |
| Lecithin | | |
| Glycerin, Sodium | Ecocertified preserver | 3% |
| Anisate, Sodium | | |
| Levulinate | | |
| Glyceril Caprilate | Ecocertified preserver | 3% |
| Disodium EDTA | Conjugator | 0.1 |
| Pentylene Glycol | Ecocertified preserver | 0.50% |
| Demineralised, sterile | Diluent | as required |
| water | | up to 100% |

* as above.

The compositions according to the present invention may be appropriately included in any of the following forms: ointment, solution, suspension, emulsion, paste, gel, cream, lotion, powder, soap, foam, surfactant-containing cleansing, oil, serum, drops, liposomes, nanoparticles, nanoboots and spray, and may be advantageously made part of a bandage.

### Example 4

A prospective, controlled trial was conducted on a group of 50 people (group A) and on a control group of 50 people (group B), male and female, different races, aged between 20 and 80 years, in order to evaluate the efficacy of the cosmetic composition.

Study Eligibility

All patients, made a complete blood examination before blood collection, including testing for viral Hepatitis, HIV, CJD and was analyzed from a clinical point of view.

Exclusion criteria were: history of cancers, pregnancy, anemia, preexisting coagulation defects including trombocitopenia, hypofibrinoginemia, anticoagulation medications, ipersensitivity to bovine product which may be used for platelet activation.

Every patient group A and B had an Informed Consent forms, skin analysis and photographs.

Study Design

The following important skin characteristics (superficial anatomy) and elements were analyzed prior and after the study protocol:
Texture (renewal and desquamation of the stratum corneum).

Surface texture of the epidermis was established in a consultation prior to starting protocol;
Colour (abnormal production of melanin resulting in hyper- or hypopigmentation) was analyzed;
Secretion (acid mantle, sebaceous and sudiferous glands) was analyzed.

Skin analysis is fundamental for measurable and visible skin results after non-invasive treatment to improve tone or use of creams. Skin surface anatomy and morphology should be studied before considering a rejuvenation option. This included analysis of dermal collagen and elastosis, in order to make reasonable predictions.

Therefore, skin diagnostic devices (such as ultrasound Dermascan, camera, Visia, magnifyng glass, Woodlamp, SD202 skin analyzer),were used to perform condition analysis and evaluate prior and after the study protocol:
Oiliness of skin
Dryness of skin, pore status
Melanin depth (dyschromia, lentigenes, sun-spots, telangiectasis, melanoma, freckles, melasma)
Epidermal and dermal atrophy
Underlying structured damage induced by photo-aging.

Ultrasound possibly can demonstrate dermal collagen, but is inconsistent on epidermal 3D morphology, skins secretions and elastin content (elastosis). For these important aspects the VISIA supplies superior images that can be quantified and used for follow up. The VISIA system allows the physician to assess wrinkles, mid-face ptosis, sagging as well as pigmentation.

We measured lipid, hydration, melanin depth and erythema levels (i.e. determine presence of lipid dry and oily skin).

The group A volunteers applied a fresh preparation of the cosmetic composition of the invention to wrinkles for 30 days twice a day.

The control group B volunteers applied the base composition without PRP, in the same planned way.

The composition was applied directly on the skin, on the whole face and on the back of both hands, for both groups.

Both the preparations were applied in a dose of a milliliter specifically in the furrow of forehead wrinkles, glabellar wrinkles, eye contours, nose-labial wrinkles and lips contours.

They were massaged with the finger tips until their complete absorption.

Safety Outcomes-Adverse Events

Adverse effects have never occurred.

It all cases a mild pleasant tingling sensation was noticed immediately after the cream application.

Safety profile is good because the patient's autologous plasma was used.

Down time is minimal, usually mild bruising, no swelling, allergies or erithemas occurred.

Follow-Up

All participants were asked to return for final control after the end of the study (90 days).

The following measured outcomes after 1 month were documented:
General skin improvement;
Improvement of tone;
Improvement of texture, pigmentation;
Plumping; and
Dermal volumetric biological filling.

Results

With the question: “Have you noticed a reduction in expression wrinkles?” the results were as follows:

100% of group A declared that they had noticed a reduction in expression wrinkles.

All patients of group B declared they had not noticed a reduction in expression wrinkles but had a good skin feeling and hydration.

In all cases of group A, we observed by using the skin analysis devices above described, that the rejuvenation process is faster after one week of application. In all cases the planned cream application schedule was respected.

Patient's compliance was optimum and we did not have poor-outcome and volunteer dissatisfaction.

Aged patients with articular alteration also agreed that the pain was decreased and the hand functionality was increased during treatment (hands).

From the above description and the above-noted examples, the advantage attained by the product described and obtained according to the present invention are evident.

1. Platelet-rich plasma for the external topical treatment of wound healing and/or skin regeneration, wherein the platelet concentration is at least 900,000 platelets/μl.

2. The platelet-rich plasma according to claim 1, wherein said platelets are autologous platelets.

3. The platelet-rich plasma according to claim 1, wherein said platelets are allogenic platelets.

4. Method for the preparation of the platelet-rich plasma according to claim 1, comprising the steps of:
a. collecting blood in a test tube;
b. separating the platelets by centrifugation;
c. mixing the platelets obtained in step b. with calcium chloride until the concentration of at least 900,000 platelets/μl is obtained.

5. A cosmetic composition comprising an effective amount of the platelet-rich plasma according to claim 1, and cosmetically acceptable excipients.

6. The cosmetic composition according to claim 5, wherein the platelet-rich plasma concentration is from 0.1 to 5.0% (w/w) of the total weight of the composition.

7. The cosmetic composition according to claim 5, being in a form selected from the group consisting of a solution, suspension, emulsion, ointment, foam, paste, gel cream, lotion, powder, soap, surfactant-containing cleansing, oil, serum, drops, liposomes, nanoparticles, nanoboots and spray.

8. The cosmetic composition according to claim 5, being impregnated or made part of a bandage.

9. The cosmetic composition according to claim 8, wherein the bandage is a surgical dressing, a plaster bandage, an adhesive bandage or a gauze.

10. A method of skin repair and regeneration and/or skin protection and/or preventing hair loss and/or for promoting the physiological growth of hair, reducing photoaging and/or skin oxidative stress and or regenerating the hair bulb cells and scalp in subjects in need thereof with the cosmetic composition according to claim 5, said method comprising treating said subjects with an effective amount of said cosmetic composition.

11. The cosmetic composition according to claim 5, further comprising a component selected from the group consisting of: monocytes, stem cells, gene therapy products, vitamins, palmitate retinol, tocoferil acetate, sodium ascorbil phosphate, D-panthenol, peptides, recombinant growth factors, micronized human-identical hormones, aminoacids, phyto-extracts, anti-oxidants, lipoic acid, DMAE, collagen, GAG, hyaluronic acid, proteoglycans, adenine, guanine, cytosine, thymine, trace elements, minerals, proteases, ceramides, polysaccarides, algae and marine extracts.

12. A pharmaceutical composition, comprising an effective amount of the platelet-rich plasma according to claim 1, and pharmaceutically acceptable excipients.

13. The pharmaceutical composition according to claim 12, wherein the platelet-rich plasma concentration is from 5.1 to 50.0% (w/w) of the total weight of the composition.

14. The pharmaceutical composition according to claim 12, being in a form selected from the group consisting of ointment, solution, suspension, emulsion, paste, gel, cream, lotion, powder, soap, foam, surfactant-containing cleansing, oil, serum, drops, liposomes, nanoparticles, nanoboots and spray.

15. The pharmaceutical composition according to claim 12, being impregnated or made part of a bandage.

16. A method of treating wound healing and/or traumatic injuries in subjects in need thereof with the pharmaceutical composition according to claim 12, said method, comprising treating said subjects with an effective amount of said pharmaceutical composition.

17. The method according to claim 16, wherein the wound is selected from the group consisting of a burn, a cut, an incision, and abrasion, a laceration and a contusion.

18. The method according to claim 17, wherein said traumatic injuries are selected from the group consisting of chronic tendinopathies, acute ligamentous injuries, muscle injuries, bone injuries, rheumatic osteoarthritis, rheumatoid disease, autoimmune diseases, articular cartilage lesions, joint degenerations, maxillofacial injuries and osteoporosis.

19. The pharmaceutical composition according to claim 12, further comprising a component selected from the group consisting of: monocytes, stem cells, gene therapy products, vitamins, palmitate retinol, tocoferil acetate, sodium ascorbil phosphate, D-panthenol, peptides, recombinant growth factors, micronized human-identical hormones, aminoacids, phyto-extracts, anti-oxidants, lipoic acid, DMAE, collagen, GAG, hyaluronic acid, proteoglycans, adenine, guanine, cytosine, thimine, trace elements, minerals, proteases, ceramides, polysaccharides, algae and marine extracts.

20-21. (canceled)
